(19) United States
(12) Patent Application Publication
(10) Pub. No.: US 2008/0188810 A1
(43) Pub. Date: Aug. 7, 2008

(54) Pump Assembly With Safety Valve

(75) Inventors: Bjorn Gullak Larsen, Birkerod (DK); Steffen Hansen, Hillerod (DK); Kristian Glejbol, Glostrup (DK); Peter Moller-Jensen, Horsholm (DK); Tue Toft, Copenhagen (DK); Niels Hvid, Vedbaek (DK); Preben Larsen, Humlebaek (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/816,729

(22) PCT Filed: Feb. 24, 2006
(86) PCT No.: PCT/EP2006/060277

§ 371 Date: Nov. 15, 2007

(30) Foreign Application Priority Data
Feb. 25, 2005 (DK) PA 2005 00290

(51) Int. Cl. A61M 5/142; A61M 5/145; A61M 1/00; F04B 19/04; F04B 19/00; F04B 19/22

(52) U.S. Cl. 604/152; 604/151; 417/440

(57) ABSTRACT

The invention provides a pump assembly comprising a suction pump and a safety valve arranged between the pump inlet and a fluid inlet for the assembly. The safety valve comprises an inlet valve and a moveable control member acting on the safety inlet valve, wherein the control member is operatable between an initial position in which the safety inlet valve is closed, and an activated position in which the safety inlet valve is open, and wherein the control member is moved from the initial to the activated position by means of suction action from the pump. By this arrangement the opening of the safety valve is positively controlled by the suction provided by the pump whereas the safety valve is closed during non-operation of the pump.

The present invention generally relates to a pump assembly comprising a safety valve adapted to prevent unintended flow of fluid through the pump assembly.

### BACKGROUND OF THE INVENTION

In the disclosure of the present invention reference is mostly made to the treatment of diabetes by injection or infusion of insulin, however, this is only an exemplary use of the present invention.

Portable drug delivery devices for delivering a drug to a patient are well known and generally comprise a reservoir adapted to contain a liquid drug and having an outlet in fluid communication with a hollow infusion needle, as well as expelling means for expelling a drug out of the reservoir and through the skin of the subject via the hollow needle. Such devices are often termed infusion pumps.

Basically, infusion pumps can be divided into two classes. The first class comprises infusion pumps which are relatively expensive pumps intended for 3-4 years use, for which reason the initial cost for such a pump often is a barrier to this type of therapy. Although more complex than traditional syringes and pens, the pump offer the advantages of continuous infusion of insulin, precision in dosing and optionally programmable delivery profiles and user actuated bolus infusions in connections with meals.

Addressing the above cost issue, several attempts have been made to provide a second class of drug infusion devices that are low in cost yet convenient to use. Some of these devices are intended to be partially or entirely disposable and may provide many of the advantages associated with an infusion pump without the attendant cost and inconveniencies, e.g. a disposable pump may be prefilled thus avoiding the need for filling or refilling a drug reservoir. Examples of this type of infusion devices are known from U.S. Pat. Nos. 4,340,048 and 4,552,561 (based on osmotic pumps), U.S. Pat. No. 5,858,001 (based on a piston pump), U.S. Pat. No. 6,280,148 (based on a membrane pump), U.S. Pat. No. 5,957,895 (based on a flow restrictor pump), U.S. Pat. No. 5,527,288 (based on a gas generating pump), or U.S. Pat. No. 5,814,020 (based on a swellable gel) which all in the last decades have been proposed for use in inexpensive, primarily disposable drug infusion devices, the cited documents being incorporated by reference. The disposable infusion devices generally comprises a mounting surface adapted for application to the skin of a subject by adhesive means, and a trans-cutaneous device adapted to be inserted through the skin of the subject, e.g. a needle or a soft cannula. The needle or the soft cannula may be insertable after the device has been arranged on the skin.

The drug reservoirs used for such infusion devices may be in the form of a “hard” reservoir (e.g. a cylinder-piston reservoir) or a flexible reservoir. The “hard” reservoir provides inherently good protection against accidental compression of the reservoir from the outside, thereby reducing the risk of unintended expelling of drug from the infusion device and into the patient when subjected to excessive forces, e.g. the patient carrying a skin-mounted infusion device may stumble or walk into a hard object, or the infusion device may be hit by an object. However, when a flexible reservoir is compressed from the outside the contained drug may be expelled through the outlet and into the patient. Although such a flexible reservoir normally will be protected by a relatively rigid housing, the housing may brake when subjected to excessive force, this allowing the flexible reservoir to be compressed and drug thereby unintentionally infused into the patient. Depending on the construction of the infusion device, a flexible reservoir may be arranged “downstream” of the expelling means, e.g. as for a gas generating pump, or “upstream” of the expelling means, e.g. as for a suction pump.

Having regard to the above-identified problems, it is an object of the present invention to provide a pump assembly comprising a safety valve adapted to prevent unintended flow of fluid through the pump assembly. It is a further object to provide a medical infusion device comprising a flexible reservoir and providing a high degree of safety of use.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect a pump assembly is provided comprising a fluid inlet and a fluid outlet, a suction pump having a pump inlet in fluid communication with the fluid inlet and a pump outlet in fluid communication with the fluid outlet. A first safety valve is arranged between the pump inlet and the fluid inlet, the first safety valve being operatable between an initial state in which the safety valve is closed, and an activated state in which the safety valve is open, the first safety valve being operated from the initial to the activated state by means of suction action from the pump. It is to be understood that by the term “safety valve” is provided a valve adapted to prevent, in its closed state, a flow of fluid through the inlet in case the inlet is pressurized from the outside.

By the above arrangement the opening of the safety valve is positively controlled by the suction provided by the pump whereas the safety valve is closed during non-operation of the pump. Advantageously, the safety valve is formed such that pressurizing of the valve from the fluid inlet side (e.g. with a pressure above the external pressure) will close the safety valve when it is in its open position.

Although the present invention advantageously can be used in combination with a flexible reservoir, it may also be used in combination with a “hard” reservoir as such a reservoir typically would have a moveable part, e.g. a piston, which potentially could be moved by external forces.

In an exemplary embodiment a pump assembly is provided comprising a fluid outlet and a fluid inlet, as well as a suction pump having a pump outlet in fluid communication with the fluid outlet and a pump inlet in fluid communication with the fluid inlet. The pump assembly further comprises a first safety valve arranged between the pump inlet and the fluid inlet, the first safety valve comprising a safety inlet valve and a moveable control member acting on the safety inlet valve, wherein the control member is operatable between an initial position in which the safety inlet valve is closed, and an activated position in which the safety inlet valve is open, the control member being moved from the initial to the activated position by means of suction action from the pump. The above definition does not imply that the control member and the safety inlet valve have to be formed as separate members as they may be formed integrally. The pump may be used to pump fluids in the form of liquids or gasses, the latter e.g. being the case during initial priming of the pump in which drug is sucked into and through the initially dry pump.

In a further exemplary embodiment the control member comprises or is associated with an actuator operatable between an initial position in which the control member is in the initial position, and an activated position in which the control member is in the activated position. The actuator comprises a first portion subjected to external pressure (e.g. atmospheric pressure which would be the normal pressure surrounding the pump assembly during use) and an opposed second portion in fluid communication with the pump inlet, whereby application of suction action from the pump moves the actuator to its activated position. The control member and the actuator may be formed integrally, or it may be to separate members (or assemblies) attached or not attached to each other.

The safety inlet valve may be in the form of e.g. a membrane valve comprising a valve seat and a valve membrane, the control member acting on the valve membrane to open the safety inlet valve, e.g. by lifting the membrane and the therein formed opening(s) free of the valve seat. The safety inlet valve may also be in the form of a valve member received in a corresponding valve seat.

As described above, the first safety valve may be controlled and actuated directly by the suction action of the pump, however, the pump assembly may also be provided with a sensor detecting actuation of the pump or the creation of suction action, wherein sensor input is used to actively control the safety valve, e.g. by an electrically energized actuator.

The suction pump may be of any desirable type, e.g. it may comprise a variable-volume pump chamber and an inlet respectively an outlet valve associated with the pump inlet respectively the pump outlet. The inlet and the outlet valves may be controlled by pressure generated in the pump chamber, e.g. in the form of membrane valves. The pump may also be in the form of a roller pump although this type of pump due to its compressed tubing normally provides a high degree of protection against undesired flow of fluid through the pump generated by outside pressure.

In case a relative vacuum arises at the fluid outlet (as may arise in a patient), fluid may be sucked through the valves of the pump assembly and e.g. into the patient. To prevent such a situation, the pump assembly may further comprise a second safety valve arranged in the fluid communication between the pump outlet and the fluid outlet, the second safety valve allowing fluid to move from the pump outlet to the fluid outlet, yet prevents fluid from being sucked through the pump assembly and into a patient.

The pump assembly may be provided in combination with a flexible reservoir containing a fluid drug in an interior thereof, where the reservoir is in fluid communication with or is adapted to be arranged in fluid communication with the fluid inlet. The reservoir may also be a distensible or elastic reservoir. The reservoir may e.g. be prefilled, user-fillable or in the form of a replaceable cartridge which again may be prefilled or fillable.

The pump assembly may further comprise an actuator for actuating the pump, or it may alternatively be adapted to cooperate with an external pump actuator. For example, the pump assembly may be provided in combination with a prefilled reservoir as a disposable unit, whereas the pump actuator may be incorporated in a durable unit adapted to be coupled to the disposable unit. The durable unit may also comprise an energy source and control electronics for operating the pump.

The pump assembly may also comprise a transcutaneous device adapted to be inserted through the skin of a subject, the transcutaneous device being arranged or adapted to be arranged in fluid communication with the fluid outlet. Alternatively, a medical assembly comprising a pump assembly as discussed above may be provided in combination with a transcutaneous device unit comprising a transcutaneous device adapted to be inserted through the skin of a subject, a mounting surface adapted for application to the skin of a subject, wherein the transcutaneous device unit and the pump assembly are adapted to be secured to each other in a situation of use, and wherein the transcutaneous device is adapted to be arranged in fluid communication with the fluid outlet. The transcutaneous device may be in the form of e.g. a needle, a soft cannula, a micro needle array, a traditional infusion set or non-invasive transdermal means, projecting from or arranged on a lower surface of a skin-mountable device in a situation of use.

In a further aspect of the invention, a pump assembly is provided comprising a flow path arranged between a fluid outlet and a fluid inlet, and a suction pump arranged in the flow path, comprising a variable-volume pump chamber and having an outlet valve in fluid communication with the fluid outlet and an inlet valve in fluid communication with the fluid inlet, wherein the inlet valve and the outlet valve have a combined opening resistance. The pump assembly further comprises a first safety valve having an outlet communicating with the exterior relative to the flow path through the pump, the first safety valve being arranged between the inlet valve and the fluid inlet. The first safety valve has an opening resistance less than the combined opening resistance of the inlet valve and the outlet valve.

By this arrangement it is provided that pressurizing of a connected drug reservoir above a certain level (i.e. above the opening pressure of the safety valve) will result in drug being “vented” from the pump, this before the pressure would open the pump inlet and outlet valves and thus resulting in drug being forced through the pump. For only a slight overpressure in the reservoir, neither of the valves would open.

To protect the pump inlet and outlet valves in case of a sudden and high rise in pressure in the reservoir, a flow restrictor may be arranged between the first safety valve and the inlet valve, such a flow restrictor having a neglectable flow resistance during normal operation of the pump but a high flow resistance during a sudden rise in flow. The latter is based on the fact that flow resistance in a conduit rises with the fourth power of the flow velocity and thus the pressure difference across the flow resistance. This is the same principle utilized in most shock absorbers. The flow restrictor may be in the form of a simple conduit portion having a length and bore providing a desired low flow resistance during normal operation of the pump, but which would ensure the necessary higher flow resistance should the pressure in the reservoir suddenly rise to high values.

In an exemplary embodiment the pump assembly further comprises a second safety valve arranged in the fluid communication between the outlet valve and the fluid outlet, the second safety valve allowing fluid to move from the outlet valve to the fluid outlet, yet prevents fluid from being sucked through the pump assembly and into a patient. The pump assembly may be provided in combination with a flexible reservoir containing a fluid drug in an interior thereof, the reservoir being in fluid communication with or adapted to be arranged in fluid communication with the fluid inlet. The pump assembly may also be provided as part of a medical assembly as discussed above.

As used herein, the term “drug” is meant to encompass any drug-containing flowable medicine capable of being passed through a delivery means such as a hollow needle in a controlled manner, such as a liquid, solution, gel or fine suspension. Representative drugs include pharmaceuticals such as peptides, proteins, and hormones, biologically derived or active agents, hormonal and gene based agents, nutritional formulas and other substances in both solid (dispensed) or liquid form. In the description of the exemplary embodiments reference will be made to the use of insulin. Correspondingly, the term “subcutaneous” infusion is meant to encompass any method of transcutaneous delivery to a subject.

### BRIEF DESCRIPTION OF THE DRAWINGS

In the following the invention will be further described with reference to the drawings, wherein

FIGS. 1-3 shows in perspective views sequences of use for a first embodiment of a drug delivery device,

FIG. 4 shows in a non-assembled state a needle unit and a reservoir unit for a further embodiment of a drug delivery device,

FIG. 5 shows an exploded view of the needle unit of FIG. 4,

FIG. 6 shows a perspective view of the needle unit of FIG. 4 in a first state,

FIG. 7 shows a perspective view of the needle carrier of FIG. 5,

FIG. 8 shows a perspective view of the needle unit of FIG. 4 in a second state,

FIG. 9 shows a side view of the needle unit of FIG. 4,

FIG. 10 shows a further perspective view of the needle unit of FIG. 4,

FIG. 11 shows perspective view of the interior of the reservoir unit of FIG. 4,

FIG. 12 shows a schematic representation of a process unit and a control unit,

FIGS. 13A and 13B show in a non-assembled respectively assembled state a cannula unit and a reservoir unit for a further embodiment of a drug delivery device.

FIG. 14 shows a schematic overview of a pump connected to a reservoir,

FIG. 15 shows an exploded view of a pump assembly,

FIG. 16 shows a cross-sectional view of the pump assembly of FIG. 11 in an assembled state and with a flow path indicated,

FIGS. 17 and 18 show partial cross-sectional views of the pump assembly of FIG. 11,

FIG. 19 shows a further schematic overview of a pump connected to a reservoir,

FIGS. 20A and 20B show schematic representations of a safety valve in a non-actuated respectively an actuated state,

FIG. 20C shows a schematic representation of a safety valve arrangement.

FIG. 21 shows an exploded view of a further pump assembly,

FIG. 22 shows the pump assembly of FIG. 21 in an assembled state,

FIG. 23 shows a stepped cross-sectional view of the pump assembly of FIG. 22,

FIG. 24 shows a stepped cross-sectional view of the pump assembly of FIG. 22 with a flow path indicated,

FIG. 25 shows a yet further schematic overview of a pump connected to a reservoir, and

FIGS. 26A and 26B show in a schematic representation a transcutaneous device in the form of a cannula and insertion needle combination.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not to an actual situation of use. The shown figures are schematic representations for which reason the configuration of the different structures as well as there relative dimensions are intended to serve illustrative purposes only.

Before turning to the present invention per se, a system suitable to be used in combination therewith will be described, the system comprising a pump unit, a patch unit adapted to be used in combination with the pump unit, and a remote control unit for wireless communication with the pump unit. However, the present invention may be used in any system or unit in which the features of the present invention would be relevant, e.g. in a conventional durable infusion pump or system.

Firstly, with reference to FIGS. 1-3 an embodiment of a medical device for drug delivery will be described focusing primarily on the directly user-oriented features. The transcutaneous device unit 2 comprises a transcutaneous device in the form of a hollow infusion device, e.g. a needle or soft cannula, and will thus in the following be termed a needle unit, however, the needle may be replaced with any desirable transcutaneous device suitable for delivery of a fluid drug or for sensing a body parameter.

More specifically, FIG. 1 shows a perspective view of medical device in the form of a modular skin-mountable drug delivery device 1 comprising a patch-like needle unit 2 (which may also be denoted a patch unit) and a reservoir unit 5. When supplied to the user each of the units are preferably enclosed in its own sealed package (not shown). The embodiment shown in FIG. 1 comprises a patch unit provided with an insertable steel needle, however, the embodiment is exemplary of how to use a patch unit with an insertable transcutaneous device, e.g. needle, cannula or sensor. In case an actual embodiment requires the patch unit to be mounted on the skin and the transcutaneous device inserted before a reservoir or other unit can be attached, it follows that the method of use would be adopted correspondingly.

The needle unit comprises a flexible patch portion 10 with a lower adhesive mounting surface adapted for application to the skin of a user, and a housing portion 20 in which a hollow infusion needle (not shown) is arranged. The needle comprises a pointed distal end adapted to penetrate the skin of a user, and is adapted to be arranged in fluid communication with the reservoir unit. In the shown embodiment the pointed end of the needle is moveable between an initial position in which the pointed end is retracted relative to the mounting surface, and an extended position in which the pointed end projects relative to the mounting surface. Further, the needle is moveable between the extended position in which the pointed end projects relative to the mounting surface, and a retracted position in which the pointed end is retracted relative to the mounting surface. The needle unit further comprises user-gripable actuation means in the form of a first strip-member 21 for moving the pointed end of the needle between the initial and the second position when the actuation means is actuated, and user-gripable retraction in the form of a second strip-member 22 means for moving the pointed end of the needle between the extended and the retracted position when the retraction means is actuated. As can be seen, the second strip is initially covered by the first strip. The housing further comprises user-actuatable male coupling means 31 in the form of a pair of resiliently arranged hook members adapted to cooperate with corresponding female coupling means on the reservoir unit, this allowing the reservoir unit to be releasable secured to the needle unit in the situation of use. A flexible ridge formed support member 13 extends from the housing and is attached to the upper surface of the patch. In use a peripheral portion 12 of the patch extends from the assembled device as the reservoir unit covers only a portion 11 of the upper surface of the patch. The adhesive surface is supplied to the user with a peelable protective sheet.

An alternative patch unit comprising an inserter mechanism for introducing a soft cannula is shown in co-owned PCT application EP2006/050410 which is hereby incorporated by reference. This alternative unit is adapted for mounting to a skin surface before the pump unit is attached, attachment of the pump unit releasing the inserter mechanism.

The reservoir unit 5 comprises a pre-filled reservoir containing a liquid drug formulation (e.g. insulin) and an expelling assembly for expelling the drug from the reservoir through the needle in a situation of use. The reservoir unit has a generally flat lower surface adapted to be mounted onto the upper surface of the patch portion, and comprises a protruding portion 50 adapted to be received in a corresponding cavity of the housing portion 20 as well as female coupling means 51 adapted to engage the corresponding hook members 31 on the needle unit. The protruding portion provides the interface between the two units and comprises a pump outlet and contact means (not shown) allowing the pump to be started as the two units are assembled. The lower surface also comprises a window (not to be seen) allowing the user to visually control the contents of the reservoir before the two units are connected.

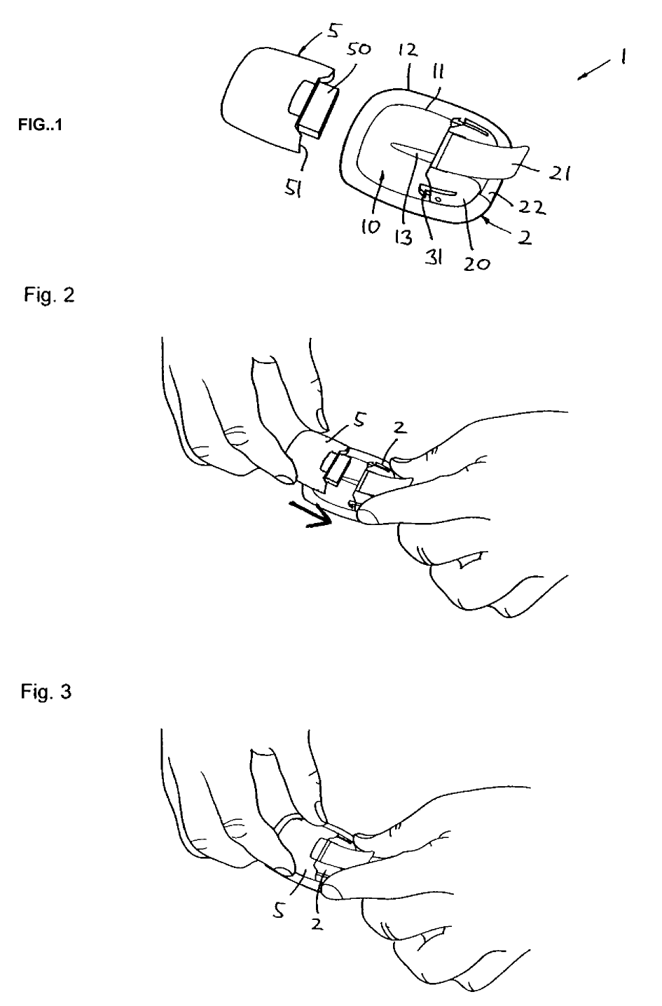

First step in the mounting procedure is to assemble the two units by simply sliding the reservoir unit into engagement with the needle unit (FIG. 2). When the hook members properly engage the reservoir unit a “click” sound is heard (FIG. 3) signalling to the user that the two units have been properly assembled. If desired, a visual or audible signal may also be generated. Thereafter the user removes the peelable sheet 14 to uncover the adhesive surface where after the device can be attached to a skin surface of the user, typically the abdomen. Infusion of drug is started by gripping and pulling away the actuation strip 21 as indicated by the arrow whereby the needle is inserted followed by automatic start of the infusion. The needle insertion mechanism may be supplied in a pre-stressed state and subsequently released by the actuation means or the needle insertion may be “energized” by the user. A “beep” signal confirms that the device is operating and drug is infused. The reservoir unit is preferably provided with signal means and detection means providing the user with an audible alarm signal in case of e.g. occlusion, pump failure or end of content.

After the device has been left in place for the recommended period of time for use of the needle unit (e.g. 48 hours)—or in case the reservoir runs empty or for other reasons—it is removed from the skin by gripping and pulling the retraction strip 22 which leads to retraction of the needle followed by automatic stop of drug infusion where after the strip which is attached to the adhesive patch is used to remove the device from the skin surface.

When the device has been removed the two units are disengaged by simultaneously depressing the two hook members 31 allowing the reservoir unit 5 to be pulled out of engagement with the needle unit 2 which can then be discarded. Thereafter the reservoir unit can be used again with fresh needle units until it has been emptied.

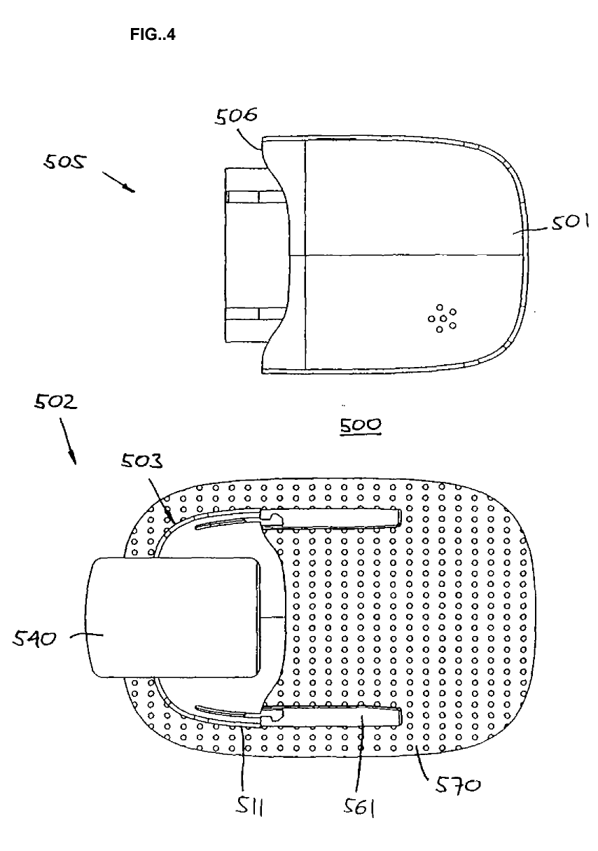

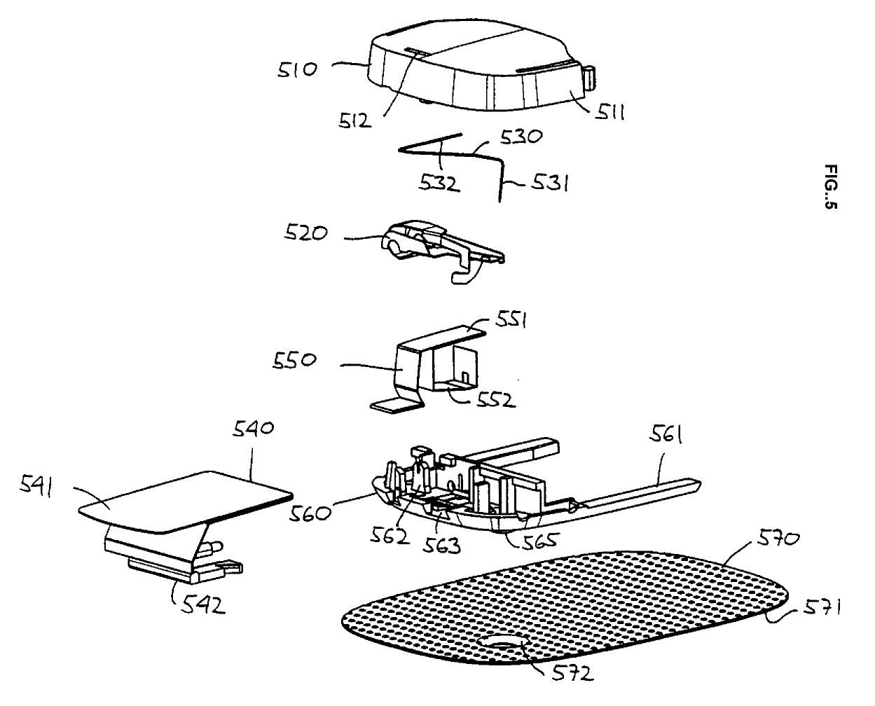

FIG. 4 shows a further embodiment of medical device 500 substantially corresponding to the embodiment of FIG. 1, the device comprising a transcutaneous device unit 502 and a process unit 505, More specifically, the transcutaneous device unit comprises a flexible patch portion (in the shown embodiment formed by a perforated sheet member 570) comprising an upper surface and a lower surface, the lower surface being adapted for application to the skin of a subject, a first housing 503 comprising a first coupling with two male coupling elements 511, and a transcutaneous device arranged in the housing (see below). Two supporting ridge members 561 extend from the first housing and are attached to the upper surface of the sheet member. The supports serve as attachment supports for the first housing, however, they may also serve to control the distance between the lower surface or the process unit and the patch. When the second unit is configured to accommodate at least partially the support members, e.g. in corresponding cut-out portions or grooves 504 (see FIG. 11), the supports may also serve to laterally stabilize the connection between the two units. The process unit comprises a second housing 501 with a lower surface and a second coupling arranged at a peripheral portion of the second housing, and a process assembly, e.g. a pump assembly as will be described below. In the shown embodiment the process unit has a generally flat rectangular shape with a cut-off end portion defining the interface with the transcutaneous device unit and also comprising the coupling in the form of two female coupling elements 506 arranged at each side of the end portion. Corresponding to FIGS. 1-3, the first and second couplings can be connected to each other with the upper surface of the patch facing towards the lower surface of the second housing. Due to the peripheral arrangement of the second coupling the flexible patch portion facing towards the lower surface of the second housing is free to move relative thereto, the degree of freedom being determined by the flexibility of the patch and supports if so provided and, of course, the surface to which the transcutaneous device unit is mounted.

In the shown embodiment the patch portion has the same general shape as the combined device albeit somewhat larger. In alternative embodiments the patch may comprise openings or cut-out portions. For example, an area between the two support legs may be cut out allowing the underlying skin to better breath.

FIG. 5 shows an exploded perspective view of the needle unit comprising an upper housing portion 510, a needle carrier 520 and a thereto mounted infusion needle 530, an actuation member 540, a release member 550, a lower housing portion 560 and a sheet member 570.

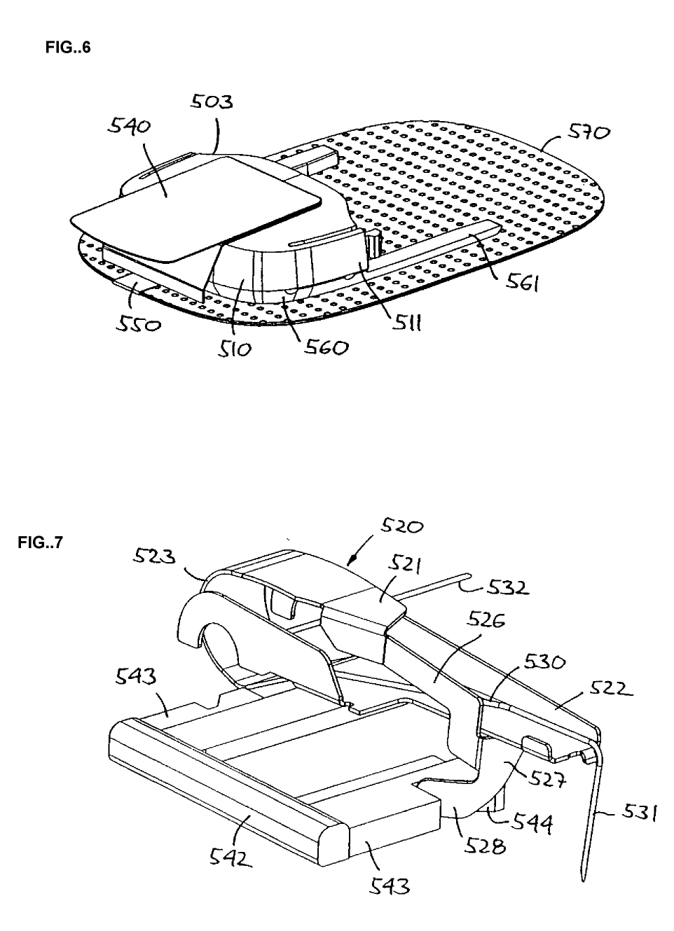

The actuation member comprises a user gripable portion 541 and a needle actuation portion 542, and the release member comprises a user gripable portion 551 and a needle retraction portion 552. In the assembled state as shown in FIG. 6, the upper and lower housing portions form a housing 503 in which the needle and the needle carrier is mounted, the actuation and release members being operatable connected to the needle carrier with the user gripable portions arranged outside the housing. The sheet member further comprises an opening 572 arranged in register with a lower optional protrusion 565 provided around the exit aperture for the transcutaneous device, just as the sheet is provided with a large number of small perforations to improve breathability through the sheet. The housing 503 is provided with user actuatable coupling means 511 allowing a reservoir unit to be attached to and released from the needle unit 505, the reservoir unit comprising corresponding mating coupling means 506 as well as a display 587. The display may indicate e.g. proper function of the unit, the amount of drug in the reservoir or different error conditions.

As seen is the user gripable portion 551 of the release member initially covered by a portion of the actuation member, this reducing the probability that the user erroneously uses the release member instead of the actuation member. Further, the actuation and release members (or portion thereof) may be colour coded to further assist the user to correctly use the device. For example, the actuation member may be green to indicate “start” whereas the release member may be red to indicate “stop”.

FIG. 7 shows in perspective the needle carrier 520 with the needle 530 and the needle actuation portion 542 of the actuation member 540. The needle actuation portion comprises two legs 543 allowing it to slide relative to the housing, the legs being arranged through respective openings 563 in the housing. The needle carrier is adapted to be connected to a hinge member 562 of the lower housing portion to thereby allow the needle carrier and thereby the needle to pivot corresponding to a pivoting axis defined by a hinge. In the shown embodiment is the needle carrier in the form a bent sheet metal member, the carrier comprising an upper arm 521 and a lower arm 522 connected to each other by a hinge portion 523 allowing the lower arm to pivot relative to the upper arm and corresponding to the pivoting axis. The lower arm forms a tray in which the hollow infusion needle 530 is mounted (e.g. by welding or adhesive), the needle having a distal pointed portion 531 adapted to penetrate the skin of the subject, the distal portion extending generally perpendicular to the mounting surface of the needle unit, and a proximal portion 532 arranged substantially corresponding to the pivoting axis and adapted to engage a fluid supply. Thus, when a portion of the upper arm is mounted in the housing, the lower arm can pivot between a first retracted position in which the distal portion of the needle is retracted within the housing, and a second extended position in which the distal portion projects relative to the mounting surface. In the shown embodiment the needle carrier provides the drive means for moving the lower arm between the two positions. This may as in the present embodiment be provided by the elastic properties of the sheet material per se corresponding to the hinge portion, or alternatively an additional spring may be provided between the two arms to thereby urge them apart. To lock the lower part in an energized, releasable first position, the upper arm is provided with a flexible release arm 526 comprising a catch 527 supporting and arresting the lower arm in its first downwardly biased position, as well as a release portion 528 engaging a ramp surface 544 of the needle actuation portion 542, the catch further comprising an inclined edge portion 529 adapted to engage the lower arm when the latter is moved from its extended to its retracted position as will be described in greater detail below.

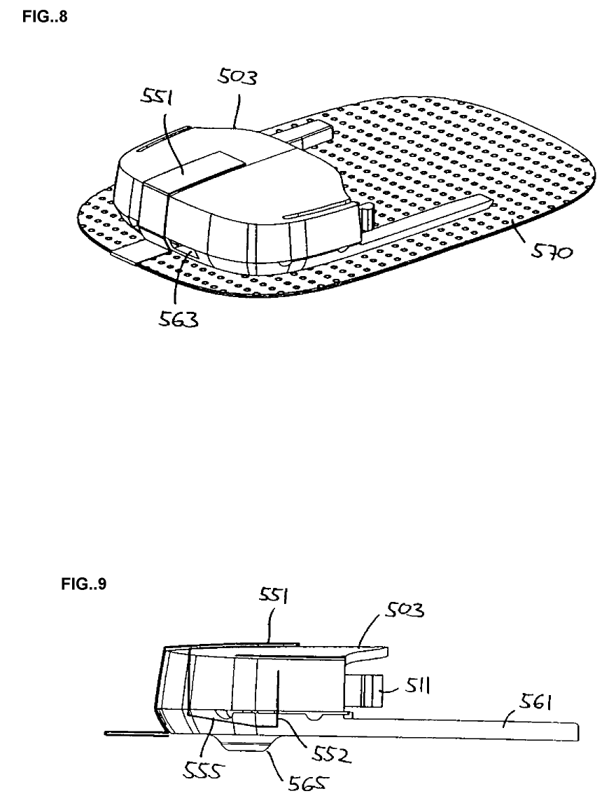

To actuate the needle the user grips the flexible strip forming the user gripable portion 541 (which preferably comprises adhesive portions to hold it in its shown folded initial position) and pulls the needle actuation portion 542 out of the housing, the actuation member 540 thereby fully disengaging the housing. More specifically, when the ramp surface 544 is moved it forces the latch 527 away from the lower arm to thereby release it, after which the release portion 528 disengages the ramp allowing the two legs to be pulled out of the housing. As seen in FIG. 8, when the actuation member is removed the user gripable portion 551 of the release member is exposed. As for the actuation member, the user gripable portion of the release member preferably comprises adhesive portions to hold it in its shown folded initial position.

In the shown embodiment the release member is in the form of a strip formed from a flexible material and having an inner and an outer end, the strip being threaded through an opening 512 in the housing, the strip thereby forming the user gripable portion 551 and the needle retraction portion 552, the inner end of the strip being attached to the housing and the outer end of the strip being attached to a peripheral portion of the sheet member 570 or, alternatively, a peripheral portion of the housing. In the projection shown in FIG. 9 the release member is shown in its initial position, the retraction portion forming a loop 555 arranged below the lower arm of the needle carrier, this position allowing the lower arm to be moved to its actuated position and thereby the needle to its extended position.

When the user decides to remove the needle unit from the skin, the user grips the user gripable portion 551, lifts it away from the housing and pulls it upwardly whereby the loop shortens thereby forcing the lower arm upwardly, this position corresponding to an intermediate release state. By this action the lower arm engages the inclined edge portion 529 of the catch 527 thereby forcing it outwardly until it snaps back under the lower arm corresponding to the position shown in FIG. 7. As the actuation member 540 has been removed from the needle unit, the needle carrier is irreversibly locked in its retracted position. When the user further pulls in the release member, the peripheral portion of the sheet member to which the release member is attached will be lifted off the skin, whereby the needle unit with its attached reservoir unit can be removed from the skin, this as described above.

Advantageously, the actuation and release members may be formed and arranged to communicate with the reservoir unit (not shown). For example, one of the legs of the actuation member may in its initial position protrude through the housing to thereby engage a corresponding contact on the reservoir unit, this indicating to the reservoir unit that the needle unit has been attached, whereas removal of the actuation member will indicate that the needle has been inserted and thus that drug infusion can be started. Correspondingly, actuation of the release member can be used to stop the pump.

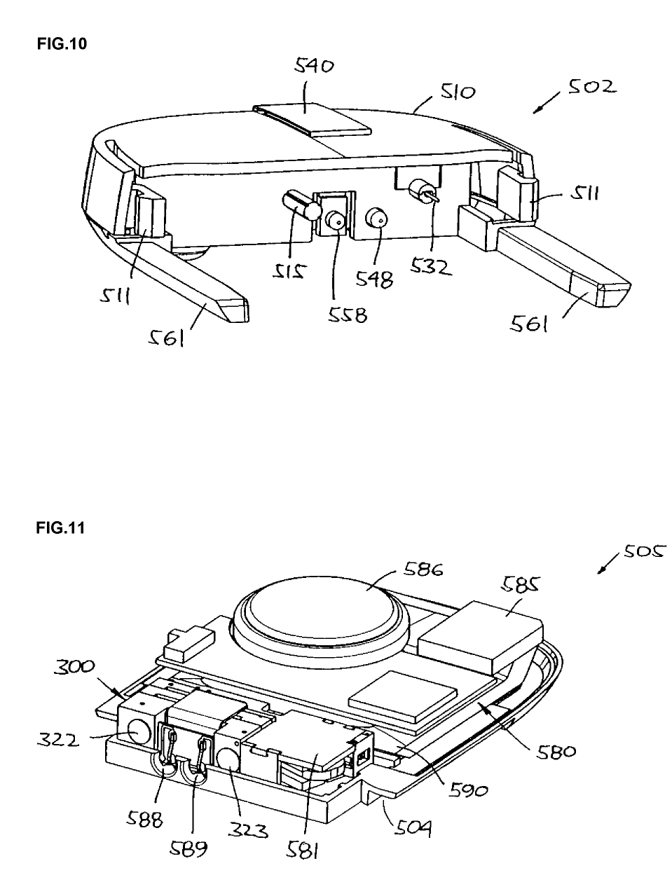

In FIG. 10 the side of the needle unit 502 which connects to the reservoir unit is shown. In addition to the two ridge members 561 and the user actuatable coupling means 511 the needle unit comprises further structures which connects to and/or engages the reservoir unit to provide a functional interface with the reservoir unit. More specifically, the needle unit comprises a fluid inlet provided by the pointed proximal portion 532 of the needle projecting from the needle unit and adapted to engage a fluid outlet of the reservoir unit, an actuator 515 projecting from the needle unit and adapted to engage and actuate a fluid connector in the reservoir unit (see below), and first and second contact actuators 548, 558 adapted to engage corresponding contacts on the reservoir unit. The first contact actuator is provided by the distal end of one of the legs 543 of the needle actuator projecting through an opening in the housing, and the second contact actuator is provided by a hinged portion of the housing connected to the needle retraction portion 552 of the release member 550. When the needle unit is first connected to the reservoir unit both contact actuators will protrude from the housing and engage the corresponding contacts on the reservoir unit thereby indicating that that a needle unit has been connected. When the needle is actuated the first contact actuator will be withdrawn and thereby disengage the corresponding contact on the reservoir unit to start pump actuation. When the needle is retracted the second contact actuator will pivot and disengage the corresponding contact on the reservoir unit to stop pump actuation.

FIG. 11 shows the reservoir unit with an upper portion of the housing removed. The reservoir unit comprises a reservoir 599 and an expelling assembly comprising a pump assembly 300 and control and actuation means 580, 581 therefore. The pump assembly comprises an outlet 322 for connection to a transcutaneous access device (e.g. the needle 530) and an opening 323 allowing an internal fluid connector to be actuated, see below. The reservoir 590 is in the form of prefilled, flexible and collapsible pouch comprising a needle-penetratable septum adapted to be arranged in fluid communication with the pump assembly, see below. The shown pump assembly is a mechanically actuated membrane pump, however, the reservoir and expelling means may be of any suitable configuration.

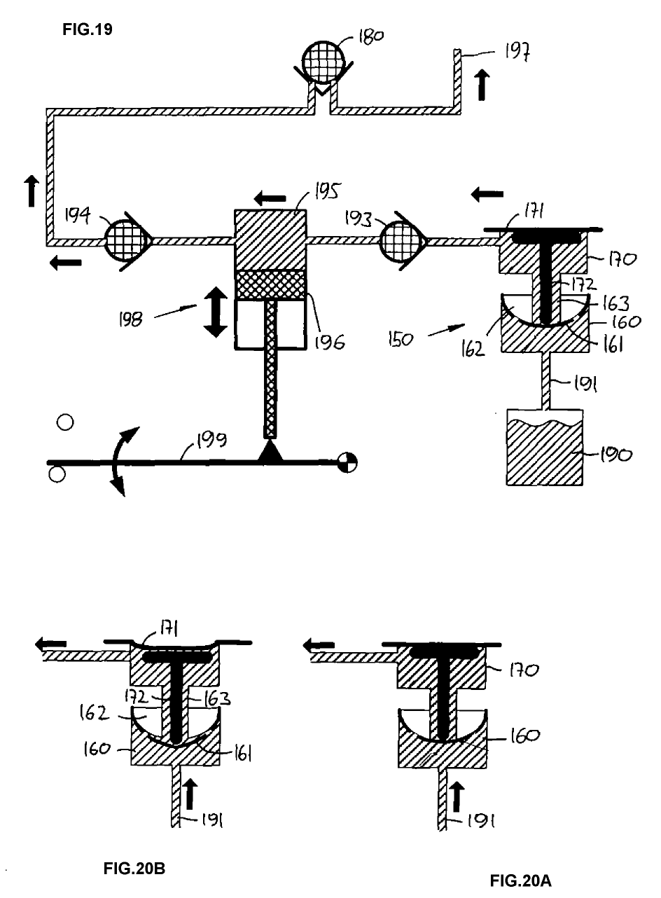

The control and actuation means comprises a pump actuating member in the form of a coil actuator 581 arranged to actuate a piston of the membrane pump via a pivoting actuation member (see FIG. 19), a PCB or flex-print to which are connected a microprocessor 583 for controlling, among other, the pump actuation, contacts 588, 589 cooperating with the contact actuators on the needle unit, signal generating means 585 for generating an audible and/or tactile signal, a display (not shown) and an energy source 586. The contacts are preferably protected by membranes which may be formed by flexible portions of the housing.

With reference to FIGS. 1-11 a modular drug delivery unit comprising a pump unit and a patch unit has been described, however, the drug delivery unit may also be provided as a unitary unit.

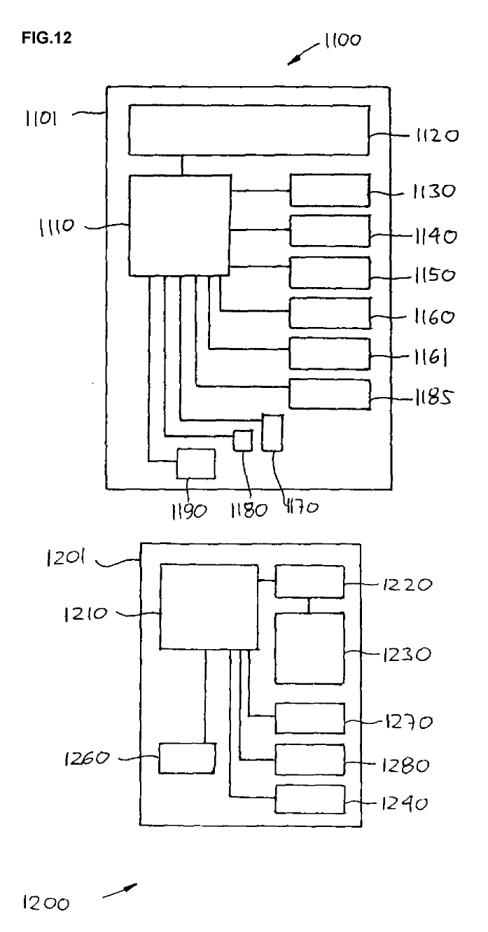

FIG. 12 shows a schematic representation of a process unit 1200 (here corresponding to the pump unit 5 of FIG. 1) and a controller unit 1100 (here in the form of a wireless “remote controller” or “external communication device” for the pump unit). It is considered that the general design of such units is well known to the skilled person, however, for a more detailed description of the circuitry necessary to provide the desired functionality of the present invention reference is made to incorporated US 2003/0065308.

More specifically, FIG. 12 depicts a simplified block diagram of various functional components or modules (i.e. single components or groups of components) included in the pump unit 1200 and remote controller 1100. The remote controller unit includes a housing 1101, a remote processor 1110 including a CPU, memory elements for storing control programs and operation data and a clock, an LCD display 1120 for providing operation for information to the user, a keypad 1130 for taking input from the user, an audio alarm 1140 for providing information to the user, a vibrator 1150 for providing information to the user, a main battery 1160 for supplying power to the controller, a backup battery 1161 to provide memory maintenance for the controller, a remote radio frequency (RF) telemetry transmitter 1170 for sending signals to the pump unit, a remote radio frequency (RF) telemetry receiver 1180 for receiving signals from the pump unit, and a second transmitter 1190. The controller further comprises a port 1185, e.g. an infrared (IR) or RF input/output system, or a USB port for communicating with a further device, e.g. a blood glucose meter (BGM), a continuous blood glucose meter (CGM), a PC or a PDA.

As also depicted in FIG. 12, the pump unit 1200 includes a housing 1201, local processor electronics 1210 including a CPU and memory elements for storing control programs and operation data, battery 1260 for providing power to the system, a process unit RF telemetry transmitter 1270 for sending communication signals to the remote unit, a process unit radio frequency (RF) telemetry receiver 1280 for receiving signals from the remote unit, a second process unit receiver 1240 (which may be in the form of a coil of an acoustic transducer used in an audio alarm for providing feedback to the user), a reservoir 1230 for storing a drug, and a pump assembly 1220 for expelling drug from the reservoir through a transcutaneous device to the body of a patient. In alternative embodiments the pump unit may also comprise an LCD display for providing information to the user, a keypad for taking input from the user, and a vibrator or other tactile actuator for providing information to the user. RF transmission may be in accordance with a standard protocol such as Bluetooth®.

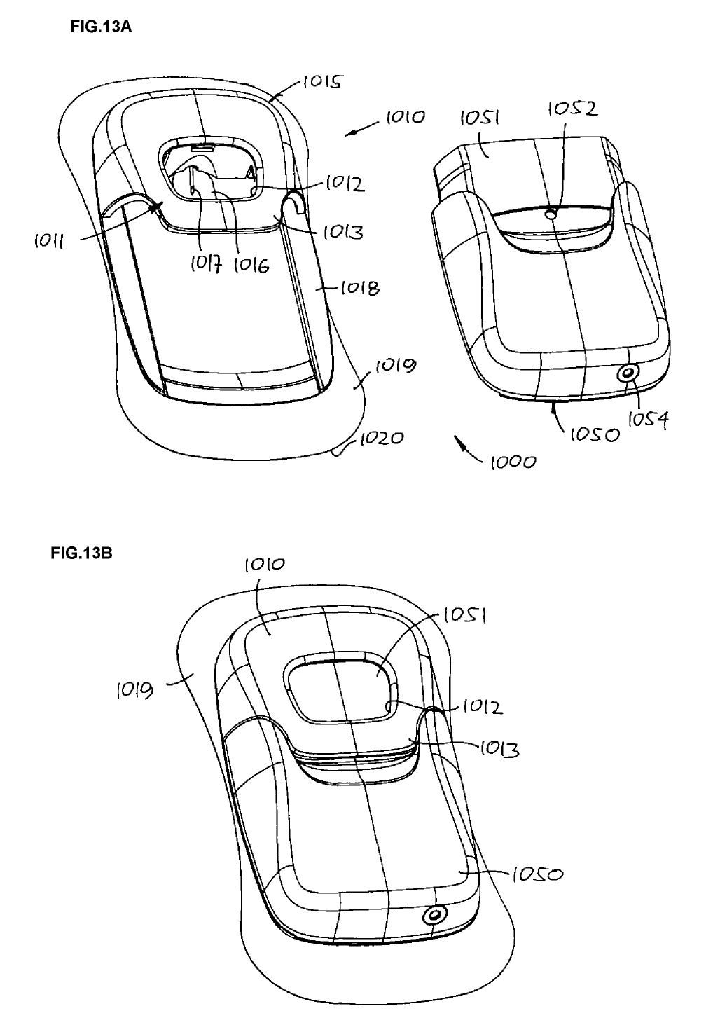

In FIG. 13A is shown an embodiment of a medical device 1000 of the type shown in FIG. 1, comprising a cannula unit 1010 and a thereto mountable pump (or reservoir) unit 1050, however, instead of a needle insertion mechanism as in the FIG. 1 embodiment, a cannula inserter mechanism as disclosed in PCT application EP2006/050410 is used. In the shown embodiment the cannula unit comprises a housing 1015 with a shaft into which a portion 1051 of the pump unit is inserted. The shaft has a lid portion 1011 with an opening 1012, the free end of the lid forming a flexible latch member 1013 with a lower protrusion (not shown) adapted to engage a corresponding depression 1052 in the pump unit, whereby a snap-action coupling is provided when the pump unit is inserted into the shaft of the cannula unit. Also a vent opening 1054 can be seen. The housing 1015 is provided with a pair of opposed legs 1018 and is mounted on top of a flexible sheet member 1019 with a lower adhesive surface 1020 serving as a mounting surface, the sheet member comprising an opening 1016 for the cannula 1017.

As appears, from the housing of the cannula unit a cannula extends at an inclined angle, the cannula being arranged in such a way that its insertion site through a skin surface can be inspected (in the figure the full cannula can be seen), e.g. just after insertion. In the shown embodiment the opening in the lid provides improved inspectability of the insertion site. When the pump unit is connected to the cannula unit it fully covers and protects the cannula and the insertion site from influences from the outside, e.g. water, dirt and mechanical forces (see FIG. 13B), however, as the pump unit is detachable connected to the cannula unit, it can be released (by lifting the latch member) and withdrawn fully or partly from the cannula unit, this allowing the insertion site to be inspected at any desired point of time. By this arrangement a drug delivery device is provided which has a transcutaneous device, e.g. a soft cannula as shown, which is very well protected during normal use, however, which by fully or partly detachment of the pump unit can be inspected as desired. Indeed, a given device may be formed in such a way that the insertion site can also be inspected, at least to a certain degree, during attachment of the pump, e.g. by corresponding openings or transparent areas, however, the attached pump provides a high degree of protection during use irrespective of the insertion site being fully or partly occluded for inspection during attachment of the pump.

In the shown embodiment an inclined cannula is used, however, in an alternative embodiment a needle mechanism of the type shown in FIG. 7 may be used if the point of insertion was moved closer to the coupling portion of the needle unit, this allowing also such a perpendicularly inserted to be inspected by detaching the pump unit.

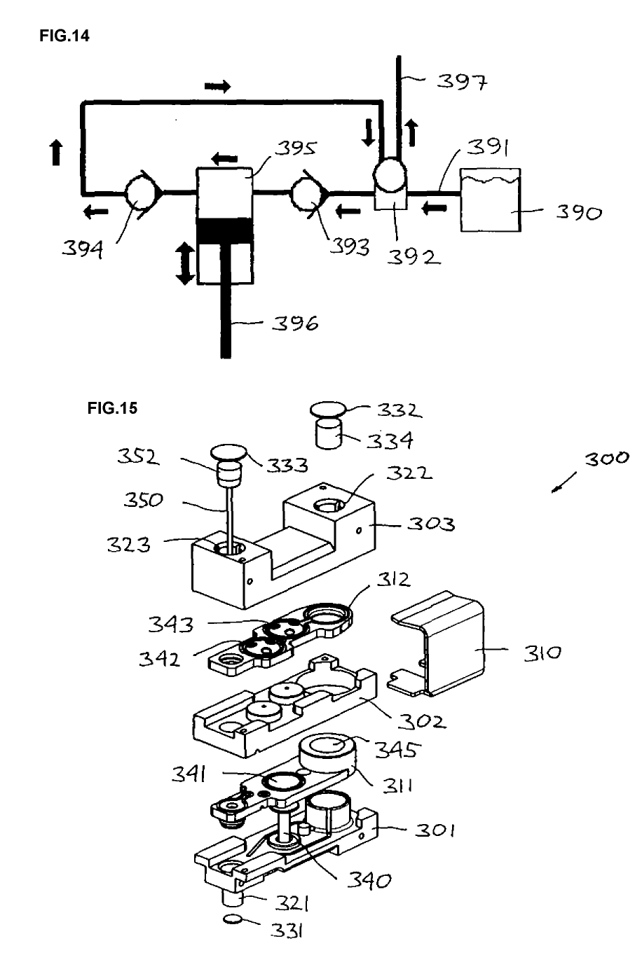

With reference to FIG. 14 a schematic overview of a pump assembly connected to a reservoir is shown, the pump assembly comprising the following general features: a fluid inlet 391 in fluid communication with a reservoir 390, a safety valve 392, a suction pump per se having inlet and outlet valves 393, 394 and a pump chamber 395 with an associated piston 396, and an outlet 397. The arrows indicate the flow direction between the individual components. When the piston is moved downwards (in the drawing) a relative negative pressure will build up inside the pump chamber which will cause the inlet valve to open and subsequently fluid will be drawn form the reservoir through the open primary side of the safety valve by suction action. When the piston is moved upwards (in the drawing) a relative overpressure will build up in the pump chamber which will cause the inlet valve to close and the outlet valve and the safety valve to open whereby fluid will flow from the pump chamber through the outlet valve and the secondary side of the safety valve to the outlet. As appears, in normal operation the safety valve allows fluid passage during both intake and expelling of fluid and is thus “passive” during normal operation. However, in case the reservoir is pressurized (as may happen for a flexible reservoir) the elevated pressure in the reservoir will be transmitted to both the primary side of the safety valve and, via the pump chamber, the secondary side of the safety valve in which case the pressure on the primary side of the safety valve will prevent the secondary side to open.

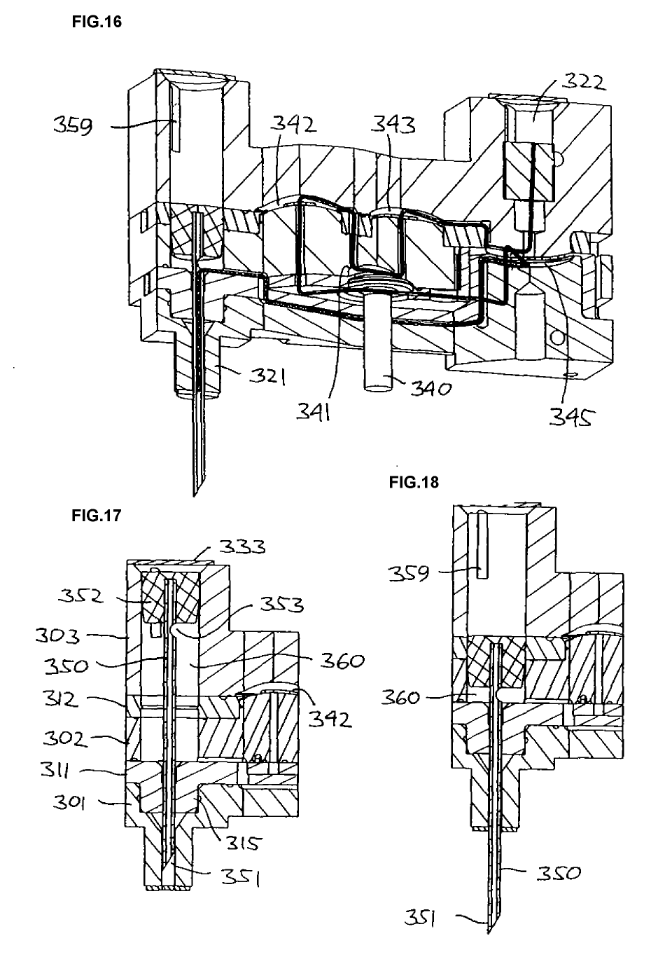

In FIG. 15 an exploded view of a pump assembly 300 utilizing the pump principle depicted in FIG. 14 is shown, the pump assembly (in the following also referred to as a pump) being suitable for use with the reservoir units of FIGS. 1-13. The pump is a membrane pump comprising a piston-actuated pump membrane with flow-controlled inlet- and outlet-valves. The pump has a general layered construction comprising first, second and third members 301, 302, 303 between which are interposed first and second membrane layers 311, 312, whereby a pump chamber 341 is formed by the first and second members in combination with the first membrane layer, a safety valve 345 is formed by the first and third members in combination with the first membrane layer, and inlet and outlet valves 342, 343 are formed by the second and third members in combination with the second membrane layer (see FIG. 16). The layers are held in a stacked arrangement by an outer clamp 310. The pump further comprises an inlet 321 and an outlet 322 as well as a connection opening 323 which are all three covered by respective membranes 331, 332, 333 sealing the interior of the pump in an initial sterile state. The membranes are penetratable or breakable (e.g. made from paper) by a needle or other member introduced through a given seal. The outlet further comprises a self-sealing, needle-penetratable septa 334 (e.g. of a rubber-like material) allowing the pump to be connected to an outlet needle. As shown in FIG. 16 a flow path (indicated by the dark line) is formed between the inlet 321 (see below) and the inlet valve 342 via the primary side of the safety valve 345, between the inlet valve, pump chamber 345 and the outlet valve 343, and between the outlet valve and the outlet 322 via the secondary side of the safety valve, the flow paths being formed in or between the different layers. The pump also comprises a piston 340 for actuating the pump membrane, the piston being driven by external driving means (not shown).

The pump further comprises a fluid connector in the form of hollow connection needle 350 slidably positioned in a needle chamber 360 arranged behind the connection opening, see FIG. 17. The needle chamber is formed through the layers of the pump and comprises an internal sealing septum 315 through which the needle is slidably arranged, the septum being formed by the first membrane layer. The needle comprises a pointed distal end 351, a proximal end on which is arranged a needle piston 352 and a proximal side opening 353 in flow communication with the distal end, the needle and the piston being slidably arranged relative to the internal septum and the chamber. As can be appreciated form FIG. 17 the needle piston in its initial position is bypassed by one or more radially placed keyways 359. These are provided in order to allow steam sterilisation and to vent the air otherwise trapped when the fluid connector is moved forward in the needle chamber.

The above-described pump assembly may be provided in a drug delivery device of the type shown in FIGS. 1-13. In a situation of use where the reservoir unit is attached to a needle unit the proximal end 532 of the infusion needle is introduced through the outlet seal and septum 334 of the pump, and the actuator 515 (see FIG. 10) is introduced through the connection membrane 333. By this action the connection needle is pushed from its initial position as shown in FIG. 17 to a actuated position as shown in FIG. 18 in which the distal end is moved through the inlet membrane 331 and further through the needle-penetratable septum of a nearby located reservoir, this establishing a flow path between the reservoir and the inlet valve via the proximal opening 353 in the needle. In this position a seal is formed between the needle piston and the needle chamber.

As appears, when the two units are disconnected, the proximal end 532 of the infusion needle is withdrawn from the pump outlet whereas the connection needle permanently provides fluid communication between the pump and the reservoir.

Turning to FIG. 19 a schematic overview of a further pump assembly connected to a reservoir is shown, the pump comprising the following general features: a fluid inlet 191 in fluid communication with a reservoir 190, a first safety valve 150, a suction pump per se 198 comprising inlet and outlet valves 193, 194 and a pump chamber 195 with an associated pump piston 196, as well as a fluid outlet 197. Further, a second safety valve 180 is arranged between the pump outlet valve and the fluid outlet. The arrows indicate the flow direction between the individual components. Apart from the safety valves the pump assembly of FIG. 19 operates in the same way as the pump assembly described with reference to FIG. 16. The first safety valve comprises in the shown embodiment a valve portion 160 (primary side) and a control portion 170 (secondary side). The valve portion comprises a valve membrane 161 with openings arranged on a valve seat 162 comprising a through-going bore 163. The valve membrane is lightly stretched to ensure proper closure during non-operation of the valve. The control portion comprises an actuator in the form of a flexible actuator membrane 171 and a control member 172 disposed between the actuator membrane and the valve membrane. The outer surface of the actuator membrane communicates with the exterior of the pump assembly (i.e. it is normally subjected to atmospheric pressure) whereas the opposed inner surface is in fluid communication with the pump inlet. The control member comprises a distal rod portion partially arranged through the valve seat bore thereby engaging the valve membrane 161, as well as a proximal plate portion engaging the inner surface of the actuator membrane, the control member thereby being in the form of a control piston. The reciprocating pump piston is actuated by a pivotally arranged actuation member 199 driven by e.g. a coil actuator.

In a non-actuated state (see FIG. 20A) the control member is merely held in place between the two membranes substantially without exerting a force thereon. Alternatively, the control member may be attached to or formed integrally with either of the two membranes. As appears, by this safety valve design there is no potential direct flow communication across the safety valve and thereby between the fluid inlet and outlet. Thus, malfunctioning of the safety valve cannot result in a fluid short circuit, this in contrast to the pump assembly of FIG. 16. Further, by detecting movement of the control member or the actuator it is possible to monitor proper functioning of the safety valve.

When the pump is operated a relative negative pressure is generated on the inner surface of the actuator membrane which is then moved inwardly by the external pressure, this in turn moving the control member towards the valve membrane which is lifted partly away from the valve seat thereby allowing flow communication through the valve membrane openings and the valve seat bore, and thus between the reservoir and the pump (see FIG. 20B). In this way the safety valve allows the pump to suck drug from the reservoir. However, when the pressure is raised in the reservoir (e.g. due to compression of a flexible reservoir), the pressure will force the valve membrane against the valve seat thereby keeping the safety valve closed. To prevent drug from being forced through the pump when the safety valve is open during normal operation, the different elements of the safety valve should be dimensioned to ensure that an open safety valve is closed by a given light overpressure in the reservoir.

The second safety valve 180 is in the form of a passive valve allowing flow of fluid out of the pump but preventing fluid from being sucked through the pump, e.g. in case a low pressure in the patient should rise. In case the second safety valve is in the form of a membrane valve, it also provides safety in a further situation of use. Normally the pump assembly and the thereto connected reservoir (e.g. a flexible reservoir) will be arranged within a housing provided with a vent. In case the vent does not function properly and the pressure outside the housing drops (as e.g. in an airplane), the pressure inside the housing will be relatively higher. As follows from this, the higher pressure in the housing will act on the secondary membrane 171 as well as the reservoir and thus open the first safety valve (due to the larger area of the secondary membrane). As the user can be considered to be “vented” to the surroundings, this would potentially result in fluid flowing from the pressurized reservoir to the user. However, as the pressure inside the housing also acts on the exterior of the second safety valve 180, fluid is prevented from flowing from the reservoir. This safety feature is further enhanced by the pressure drop through the pump and the pre-tensioning of the membrane of the second safety valve.

With reference to FIG. 20C an alternative configuration for an inlet safety valve 1150 of the type shown in FIG. 20A will be described. The safety valve has a general layered construction comprising first, second and third members 1301, 1302, 1303 between which are interposed the different moveable safety valve components, the members defining a fluid inlet 1191, a fluid outlet 1192 connected to a pump inlet valve 1193, and a ventilation opening 1175. The safety valve further comprises a safety inlet valve in the form of a conical valve member 1161 arranged in a corresponding valve seat 1162, a control member 1172 arranged through a bore in the valve seat, and a flexible actuator membrane 1171. The safety inlet valve, the control member and the actuator membrane are formed as a unitary member from an elastic polymeric material. Advantageously, such a member may be formed integrally with one of the membrane layers 311, 312. As the conical valve member 1161 is elastic, it may be threaded through the valve bore during assembly. As appears from FIG. 20C, the actuator membrane is in its initial position arranged in a pre-stressed condition over a convex portion of the ventilation opening 1175, this assuring proper positioning. In the initial position the control member is slightly stressed thereby pulling the valve member into contact with the valve seat. When the control member is moved inwardly during a pump suction stroke, the control member relaxes and subsequently opens the safety inlet valve.

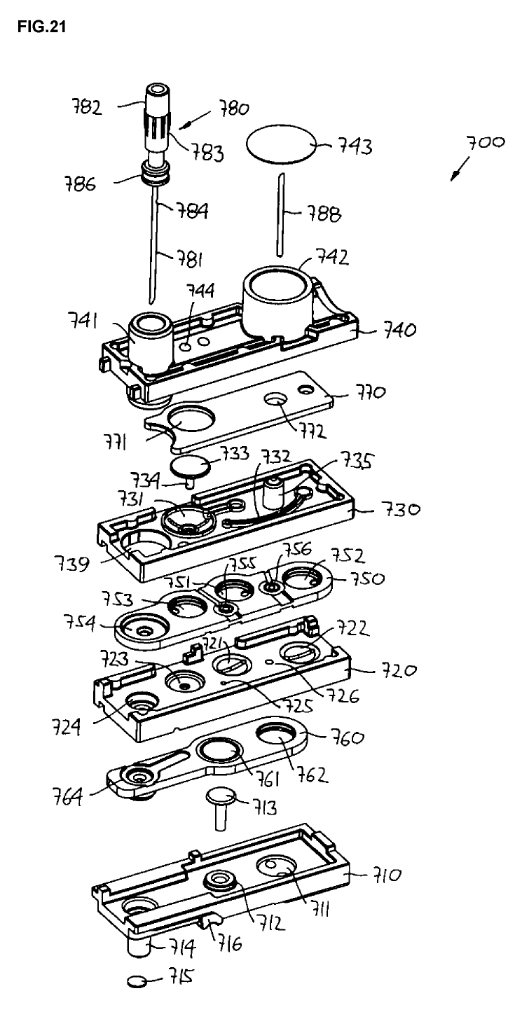

In FIG. 21 an exploded view of a pump assembly 700 utilizing the pump principle depicted in FIG. 19 is shown, the pump assembly (in the following also referred to as a pump) being suitable for use with the reservoir units of FIGS. 1-13. The pump is a membrane pump comprising a piston-actuated pump membrane with flow-controlled inlet- and outlet-valves. The pump has a general layered construction comprising four plate members, a bottom plate 710, a middle plate 720, a lower top plate 730 and an upper top plate 740, between which are interposed (in order from the bottom) second, first and third membranes 750, 760, 770. The layers are held together with clamp members 791, 792. Corresponding to the FIG. 15 embodiment, the pump also comprises a connection needle assembly 780, as well as an outlet needle 788. When in the following valves are described, these have a general configuration comprising a valve seat and an opposed valve housing between which a valve membrane is arranged.

Turning to the individual components, the bottom plate 710 comprises a housing 711 for a second safety valve (corresponding to the safety valve 180 in FIG. 19), an opening 712 through which a pump piston 713 for actuating a pump membrane is arranged, a tubular needle shroud 714 provided with needle-penetratable barrier member 715, and a bearing 716 for 716 for an actuator member. The middle plate 720 comprises an inlet valve housing 721, an outlet valve housing 722, a housing 723 for a first safety valve, a bore 724 for the connection needle, a bore 725 leading to a pump chamber housing 727 on the lower surface of the middle plate, and a bore 726 for the outlet needle 788, and, also on the lower surface, a valve seat 728 for the second safety valve. The lower top plate 730 comprises a housing 731 with a bore for a control piston, a curved conduit portion 732, a control piston 733 with a stem 734 arranged in the said bore, a tubular mounting post 735 for the outlet needle, and, on the lower surface, a valve seat 736 for the first safety valve, an inlet valve seat 737, an outlet valve seat 738, and a bore for a tubular housing portion 741. The upper top plate 740 comprises the tubular housing portion 741 forming a chamber for the connection needle assembly, a tubular shroud 742 for the outlet needle provided with a needle-penetratable barrier member 743, and vent openings 744 for a secondary safety valve membrane 771.

The second membrane 760 comprises a pump membrane 761 engaging the pump piston 713, a second safety valve membrane 762 engaging the valve seat 728, and a first septum 764 for the connection needle. The first membrane 750 comprises an inlet valve membrane 751 engaging the inlet valve seat 737, an outlet valve membrane 752 engaging the outlet valve seat 738, an a first safety valve membrane 753 engaging the safety valve seat 736, a second septum 754 for the connection needle, a bore 755 connecting the pump chamber with the curved conduit 732, and a bore 756 for the outlet needle. The third membrane 770 comprises a secondary safety valve membrane 771 for the first safety valve adapted to engage the control piston 733, and a bore 772 for the post 735. The connection needle assembly 780 comprises a hollow tubular needle 781 with a side opening 784, the needle being mounted in a hub portion 782 provided with combined locking and venting ribs 783, the proximal end of the needle being sealed by a sealing material 785. In its initial position the hub is adapted to create a seal between a hub seal member 786 and the inner surface of the tubular housing portion 741.

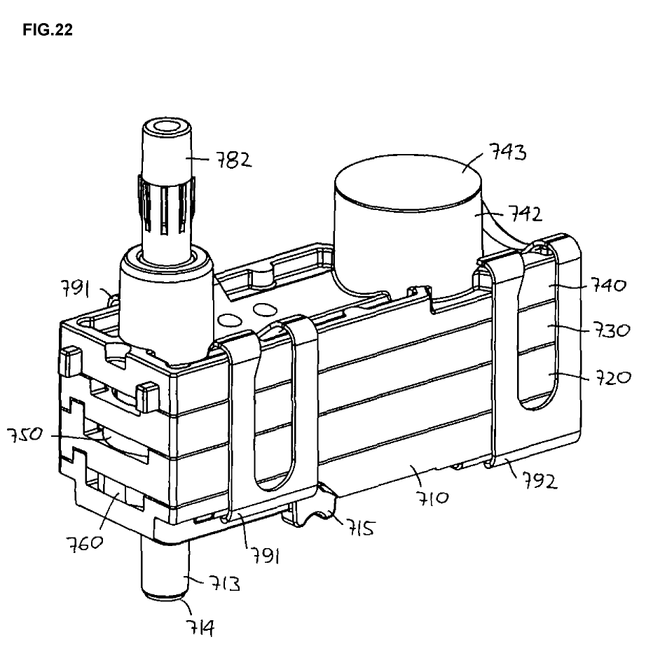

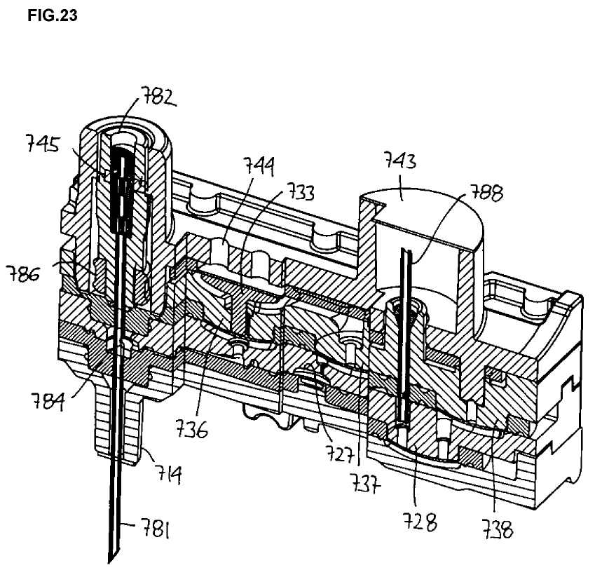

In FIGS. 22 and 23 are shown the pump in an assembled state, whereby the different valves described above, as well as the two needles, can be seen. The outlet needle 788 is fixedly mounted in fluid communication with the outlet of the second safety valve, the pointed distal outlet end allowing the pump to connected to an outlet means (e.g. a patch unit) comprising a needle-penetratable septum, this in contrast to the FIGS. 10-11 embodiment in which the needle 532 is carried by the patch unit 502. Indeed, the needle-septum connector could be replaced by a suitable needle-less connector. In the initial position (see FIG. 22) the connection needle is arranged with its distal pointed end inside the needle shroud 714 and with the needle positioned through the two self-sealing, needle-penetratable septa 754, 764. In this position the connection needle is arranged within the sterile interior of the pump sealed by the two barrier members 715, 734 and the needle hub 782. When the connection needle is actuated (see also the description of the FIGS. 16-18 embodiment), a support member 745 in the tubular housing 741 breaks away (for illustrative purposes the member 745 is shown superposed on the hub 782 in FIG. 23), and the distal end of the needle is pushed through the barrier member 715 and into a reservoir, the side opening being arranged in the bore 724 between the two septa. As the body of the needle hub has a smaller diameter than the sealing member 786, air is allowed to leave the tubular housing portion 741 as the needle hub is inserted. As appears, when the two units are disconnected, the outlet needle is withdrawn from the patch whereas the connection needle permanently provides fluid communication between the pump and the reservoir.

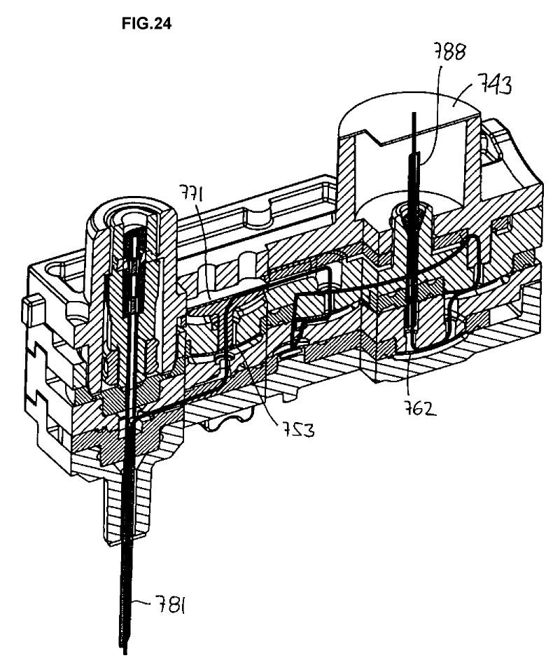

In FIG. 24 the flow path through the activated pump can be seen. From the connection needle fluid is sucked through the first safety valve and the inlet valve and into the pump chamber. From the pump chamber the fluid is pumped through the outlet valve via the curved conduit 732 and through the second safety valve and to the outlet needle.

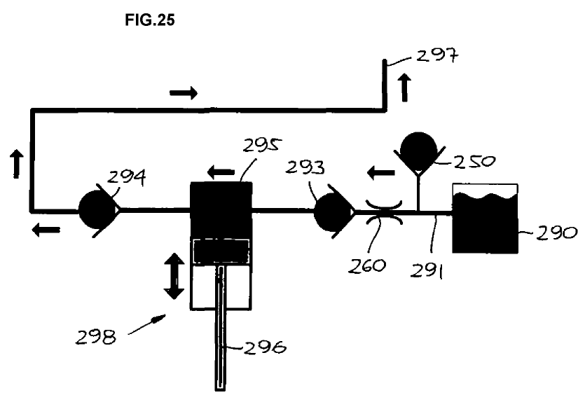

Turning to FIG. 25 a schematic overview of a further pump assembly connected to a reservoir is shown, the pump comprising the following general features: a fluid inlet 291 in fluid communication with a reservoir 290, a safety valve 250, a flow restrictor 260, a pump per se comprising inlet and outlet valves 293, 294 and a pump chamber 295 with an associated piston 296, as well as a fluid outlet 297. The arrows indicate the flow direction between the individual components. Apart from the safety valves the pump assembly of FIG. 19 operates in the same way as the pump assembly described with reference to FIG. 14. The safety valve is arranged between the inlet valve and the fluid inlet and is in the form of an outlet valve communicating with the exterior relative to the flow path through the pump, e.g. the outside of the pump assembly. The safety valve has an opening resistance less than the combined opening resistance of the inlet valve and the outlet valve, whereby it is provided that pressurizing of the drug reservoir above a certain level (i.e. above the opening pressure of the safety valve) will result in the drug being “vented” from the pump before the pressure would open the pump inlet and outlet valves and thus resulting in drug being forced through the pump and out through the fluid outlet to the patient. For only a slight overpressure in the reservoir, neither of the valves would open.

To protect the pump inlet and outlet valves in case of a sudden and high rise in pressure in the reservoir, a flow restrictor is arranged between the safety valve and the inlet valve, the flow restrictor having a neglectable flow resistance during normal operation of the pump but a high flow resistance during a sudden rise in pressure and flow. The flow restrictor may be in the form of a simple conduit portion having a length and bore providing a desired low flow resistance during normal operation of the pump, but which would ensure the necessary higher flow resistance should the pressure in the reservoir suddenly rise to high values. The embodiment of FIG. 25 may be provided with a second safety valve corresponding to the second safety valve 180 of the FIG. 19 embodiment.

In the above described embodiments, the transcutaneous device has been in the form of a unitary needle device (e.g. an infusion needle as shown or a needle sensor (not shown)), however, the transcutaneous device may also be in the form of a cannula or a sensor in combination with an insertion needle which is withdrawn after insertion thereof. For example, the first needle portion may be in the form of a (relatively soft) infusion cannula (e.g. a Teflon® cannula) and a there through arranged removable insertion needle. This type of cannula needle arrangement is well known from so-called infusion sets, such infusion sets typically being used to provide an infusion site in combination with (durable) infusion pumps.

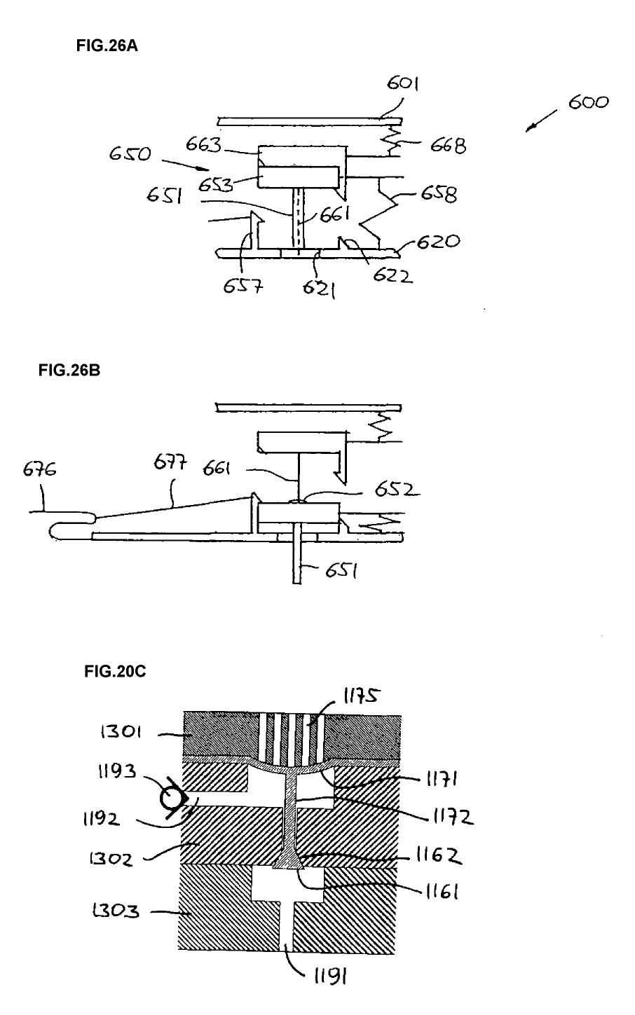

Thus, FIGS. 26A and 26B show in a schematic representation how a cannula and insertion needle combination can be arranged within a housing 601 of in a given medical device 600 (partly shown), e.g. an infusion device or an infusion set. More specifically, the medical device comprises a transcutaneous assembly 650 comprising a combination of a relatively soft cannula 651 (which e.g. may be of the soft “Teflon®” type) carried by a lower member 653 and a pointed insertion needle 661 (e.g. made from medical grade stainless steel) slidably arranged within the cannula and carried by an upper member 663, both members being mounted to allow axial displacement of the cannula respectively the insertion needle. The cannula comprises a proximal inlet (not shown) allowing it to be or to be arranged in fluid communication with a fluid source. The medical device further comprises a base plate 620 with an opening 621 for the cannula as well as a release member 622. The lower member comprises an elastomeric seal 652 through which the insertion needle is arranged. The cannula and the insertion needle may be straight or curved dependent upon how the two members are mounted in the device, e.g. arcuate corresponding to a pivoting axis or straight corresponding to linear movement as illustrated. The upper member comprises a coupling member 667 locking the members together in an initial position with distal end of the insertion needle extending from the distal opening of the cannula as shown in FIG. 26A, and the base plate comprises coupling member 657 for locking the lower member in an extended position with distal end of the cannula extending through the opening in the base plate (see FIG. 26B). Between the housing of the device and the upper member a first spring 668 is arranged biasing the upper member upwards. Correspondingly, the device also comprises a second spring 658 biasing the lower member upwardly. The medical device further comprises a gripping tab 676 and a pulling member 677 corresponding to the embodiment shown in FIG. 1.

In a situation of use the assembly is moved downwardly, either manually or by a releasable insertion aid, e.g. a spring loaded member acting through an opening in the housing (not shown) whereby the cannula with the projecting insertion needle is inserted through the skin of a subject. In this position the lower member engages the coupling member 657 to thereby lock the cannula in its extended position, just as the coupling member 667 is released by the release member 622 thereby allowing the upper member to return to its initial position by means of the first spring. When the user intends to remove the delivery device from the skin surface, the user grips the gripping portion of the tab and pulls it in a first direction substantially in parallel with the skin surface, by which action the flexible strip 677 releases the coupling member 657 from the lower member whereby the lower member and thereby the cannula is retracted by means of the second spring. When the cannula has been withdrawn from the skin, the user uses the now unfolded tab to pull off the entire delivery device from the skin surface, for example by pulling the tab in a direction away from the skin surface. A further cannula inserter mechanism is disclosed in PCT application EP2006/050410.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A pump assembly, comprising:
a fluid inlet (191) and a fluid outlet (197),
a suction pump (198) having a pump inlet in fluid communication with the fluid inlet and a pump outlet in fluid communication with the fluid outlet,
a first safety valve (150) arranged between the pump inlet and the fluid inlet,
wherein the first safety valve is operatable between an initial state in which the safety valve is closed, and an activated state in which the safety valve is open, the first safety valve being operated from the initial to the activated state by means of suction action from the pump.

2. A pump assembly as in claim 1, wherein the first safety valve comprises a safety inlet valve and a moveable control member (172) acting on the safety inlet valve, and
wherein the control member is operatable between an initial position in which the safety inlet valve is closed, and an activated position in which the safety inlet valve is open, the control member being moved from the initial to the activated position by means of suction action from the pump.

3. A pump assembly as in claim 2, wherein the control member comprises or is associated with an actuator (171) operatable between an initial position in which the control member is in the initial position, and an activated position in which the control member is in the activated position, the actuator comprising a first portion subjected to external pressure and an second portion in fluid communication with the pump inlet, whereby application of suction action from the pump moves the actuator to its activated position.

4. A pump assembly as in claim 2, wherein the safety inlet valve is in the form of a membrane valve comprising a valve seat (162) and a valve membrane (161), the control member acting on the valve membrane to open the safety inlet valve.

5. A pump assembly as in claim 1, wherein the suction pump comprises a variable-volume pump chamber (195) and an inlet respectively an outlet valve (193, 194) associated with the pump inlet respectively the pump outlet.

6. A pump assembly as in claim 5, wherein the inlet and the outlet valves are controlled by pressure generated in the pump chamber.

7. A pump assembly as in claim 1, further comprising a second safety valve (180) arranged in the fluid communication between the pump outlet and the fluid outlet, the second safety valve allowing fluid to move from the pump outlet to the fluid outlet, yet prevents fluid from being sucked through the pump assembly via the fluid outlet.

8. A pump assembly as in claim 1, further comprising a flexible reservoir (190) containing a fluid drug in an interior thereof, the reservoir being in fluid communication with or adapted to be arranged in fluid communication with the fluid inlet.

9. A pump assembly as in claim 1, further comprising an actuator (581) for actuating the pump.

10. A pump assembly as in claim 1, further comprising a transcutaneous device (530, 1017) adapted to be inserted through the skin of a subject, the transcutaneous device being arranged or adapted to be arranged in fluid communication with the fluid outlet.

11. A medical assembly comprising a pump assembly (5, 1050) as in claim 1, further comprising a transcutaneous device unit (2, 1010) comprising:
a transcutaneous device (530, 1017) adapted to be inserted through the skin of a subject,
a mounting surface (10, 1020) adapted for application to the skin of a subject,
wherein the transcutaneous device unit and the pump assembly are adapted to be secured to each other in a situation of use, and
wherein the transcutaneous device is adapted to be arranged in fluid communication with the fluid outlet.

12. A pump assembly, comprising:
a fluid inlet (291) and a fluid outlet (292),
a suction pump (298) comprising a variable-volume pump chamber and having an inlet valve (293) in fluid communication with the fluid inlet and an outlet valve (294) in fluid communication with the fluid outlet, the inlet valve and the outlet valve having a combined opening resistance,
a first safety valve (250) arranged between the inlet valve and the fluid inlet, the first safety valve having an opening resistance less than the combined opening resistance of the inlet valve and the outlet valve.

13. A pump assembly as in claim 12, comprising a flow restrictor (260) arranged between the first safety valve and the inlet valve, the flow restrictor having a neglectable flow resistance during normal operation of the pump.

14. An assembly as in claim 12, further comprising a second safety valve arranged in the fluid communication between the outlet valve and the fluid outlet, the second safety valve allowing fluid to move from the outlet valve to the fluid outlet, yet prevents fluid from being sucked through the pump assembly via the fluid outlet.
